(19) United States
(12) Patent Application Publication
(10) Pub. No.: US 2024/0398094 A1
(43) Pub. Date: Dec. 5, 2024

(54) CONSUMER PRODUCT CONTAINING VOLATILE COMPOSITION CARTRIDGE

(71) Applicant: The Procter & Gamble Company, Cincinnati, OH (US)

(72) Inventors: Rahul VYAS, Singapore (SG); Bhavesh RAJWANI, Singapore (SG); Walter SORDO, Trento (IT); Stefano BALDESSARI, Caldonazzo (IT)

(21) Appl. No.: 18/734,295

(22) Filed: Jun. 5, 2024

Related U.S. Application Data
(60) Provisional application No. 63/567,472, filed on Mar. 20, 2024.
(60) Provisional application No. 63/566,976, filed on Mar. 19, 2024.
(60) Provisional application No. 63/611,920, filed on Dec. 19, 2023.
(60) Provisional application No. 63/470,993, filed on Jun. 5, 2023.

(51) Int. Cl. A45D 34/02; A61L 9/012

(52) U.S. Cl. CPC A45D 34/02; A61L 9/012

(57) ABSTRACT

A consumer product includes an outer packaging; one or more cartridges containing a volatile composition; and instructions for using the one or more cartridges with a reusable housing. The one or more cartridges are packaged within the outer packaging, and each include: a reservoir for interfacing with the reusable housing, the reservoir containing a volatile composition in liquid form and having an opening; a membrane enclosing the opening of the reservoir, the membrane configured to allow evaporation of the volatile composition; and a sealing substrate enclosing the opening of the reservoir. The one or more cartridges are single-use cartridges for placing into the reusable housing; and the one or more cartridges are configured to be placed into the reusable housing without adhesion or attachment to the reusable housing.

### TECHNICAL FIELD

The present disclosure relates to the field of consumer products, and specifically to products comprising cartridges for delivering a volatile composition.

### BACKGROUND

Systems for delivering volatile materials to the atmosphere are well known in the art. Such systems include insect repellents, air fresheners, malodor removal agents, or the like, and function by evaporating a volatile material into a space to deliver a variety of benefits such as air freshening or malodor removal.

Most volatile composition dispensers are configured for one-time use. Typical disposable air freshener dispenser devices are described in PCT Publication No. WO 98/16262 and WO2017/192639, which include an air freshener medium within a container, and a push button actuator which can be manually operated to rupture a foil covering the container for initiating the dispensing of the air freshener into the atmosphere. A problem associated with such device is that after volatile composition is depleted, it would not be possible to refill or replenish the volatile composition and reactivate the volatile composition dispenser. As a result, the entire product is disposed of, contributing to the environmental problem of plastics waste.

Therefore, there exists a need for a volatile composition dispenser with a reusable housing and a replaceable cartridge therein for delivering a volatile material.

Furthermore, there exists a need for a volatile composition dispenser that is easy to use and provides high consumer satisfaction.

### SUMMARY

The present disclosure provides the following.

1. A consumer product comprising:
(i) an outer packaging;
(ii) one or more cartridges containing a volatile composition; and
(iii) instructions for using the one or more cartridges with a reusable housing;
wherein the one or more cartridges are packaged within the outer packaging, and each comprise:
   a reservoir for interfacing with the reusable housing, the reservoir containing a volatile composition in liquid form and having an opening;
   a membrane enclosing the opening of the reservoir, the membrane configured to allow evaporation of the volatile composition; and
   a sealing substrate enclosing the opening of the reservoir, wherein:
the one or more cartridges are single-use cartridges for placing into the reusable housing; and
the one or more cartridges are configured to be placed into the reusable housing without adhesion or attachment to the reusable housing; and
either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

2. A consumer product comprising:
(i) an outer packaging;
(ii) one or more cartridges containing a volatile composition; and
(iii) instructions for using the one or more cartridges with a reusable housing;
wherein the one or more cartridges are packaged within the outer packaging, and each comprise:
   a reservoir for interfacing with the reusable housing, the reservoir containing a volatile composition in liquid form and having an opening;
   a membrane enclosing the opening of the reservoir, the membrane configured to allow evaporation of the volatile composition; and
   a sealing substrate enclosing the opening of the reservoir, wherein:
the one or more cartridges are single-use cartridges for placing into the reusable housing; and
the one or more cartridges are configured to be removed from the reusable housing without being touched by a user; and
either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

3. The consumer product according to clause 1 or 2, wherein the sealing substrate encloses the membrane, and the sealing substrate is configured to be removed before use

4. The consumer product according to any one of the preceding clauses, wherein the one or more cartridges comprise an outer peripheral seal area, and wherein a peripheral portion of the sealing substrate and membrane are sealed together at the outer peripheral seal area, wherein the membrane is sealed to a peripheral portion of the reservoir at the outer peripheral seal area.

5. The consumer product according to any one of the preceding clauses, wherein the sealing substrate comprises a pull tab to facilitate removal by a user.

6. The consumer product according to any one of the preceding clauses, wherein the volatile composition comprises a perfume.

7. The consumer product according to any one of the preceding clauses, wherein the volatile composition comprises a volatile carbonyl containing compound having a vapor pressure of at least 0.025 torr at 25 degrees Celsius, wherein the volatile carbonyl containing compound is selected from the group consisting of: volatile aldehydes, ketones, and mixtures thereof.

8. The consumer product according to any one of the preceding clauses, wherein the one or more cartridges comprise at least one secondary reservoir portion, wherein when the cartridge is oriented for use:
at least one secondary reservoir portion is gravitationally below the reservoir; and
a total volume of the at least one secondary reservoir portion that is gravitationally below the primary reservoir portion is at least 4% of the initial volume of volatile composition.

9. The consumer product according to any one of the preceding clauses, wherein the reservoir is an outermost layer of the one or more cartridges.

10. The consumer product according to any one of the preceding clauses, wherein the one or more cartridges are compressible or deformable.

11. The consumer product according to clause 10, wherein the membrane and reservoir are each compressible or deformable.

12. The consumer product according to any one of the preceding clauses, wherein the membrane has an evaporative surface area of from about 20 cm² to about 40 cm², and the one or more cartridges have a maximum dimension of less than 10 cm.

13. The consumer product according to any one of the preceding clauses, wherein the membrane forms substantially all of the surface area of a face of the cartridge, wherein the membrane forms at least 90% of the surface area of a face of the cartridge, wherein the membrane forms at least 95% of the surface area of a face of the cartridge.

14. The consumer product according to any one of the preceding clauses, wherein the reservoir is formed from a transparent material to enable viewing of a fill level of volatile composition.

15. The consumer product according to any one of the preceding clauses, wherein the reusable housing comprises one or more rib elements that are configured to apply a compressive force to a cartridge placed within the reusable housing, where the compressive force is configured to be applied to the cartridge for substantially as long as the cartridge is present within the reusable housing.

16. The consumer product according to any one of the preceding clauses, wherein the reusable housing comprises a front frame and a rear frame that are capable of moving away from or toward each other for opening and closing the reusable housing, wherein:
the front frame comprises a window configured to receive a part of the cartridge (e.g. the reservoir);
at least a part of a perimeter of the window is bordered by a wall portion protruding substantially perpendicular from the window towards an interior of the reusable housing, the wall portion configured to support the cartridge (e.g. the reservoir) within the reusable housing without adhesion;
the rear frame comprises one or more apertures configured to allow an airflow into the reusable housing; and
the reusable housing is configured to hold the cartridge between the front and rear frames so as to allow the volatile composition to evaporate from the cartridge.

17. The consumer product according to any one of the preceding clauses, wherein the reusable housing comprises a front frame and a rear frame that are capable of moving away from or toward each other for opening and closing the reusable housing, wherein the rear frame comprises one or more rib elements that are configured to apply a compressive force on the cartridge when the reusable housing is in a closed state and the cartridge is positioned within the reusable housing, where the compressive force is configured to be applied to the cartridge for substantially as long as the cartridge is present within the reusable housing.

18. The consumer product according to clause 17, wherein the compressive force is configured to cause a reduction in a thickness of the reservoir of the cartridge when held within the reusable housing, as compared to a thickness of the reservoir before being subjected to the compressive force.

19. A consumer product comprising:
(i) an outer packaging; and
(ii) one or more cartridges containing a volatile composition, the one or more cartridges being single-use cartridges for placing into a reusable housing and packaged within the outer packaging, and each comprising:
   a reservoir for interfacing with the reusable housing, the reservoir containing a volatile composition in liquid form and having an opening;
   a membrane enclosing the opening of the reservoir, the membrane configured to allow evaporation of the volatile composition; and
   a sealing substrate enclosing the opening of the reservoir, wherein:
the one or more cartridges are configured such that, when placed into a housing comprising a front frame and a rear frame that are capable of moving away from or toward each other for opening and closing the housing, wherein: the front frame comprises a window configured to receive a part of the cartridge (e.g. the reservoir); at least a part of a perimeter of the window is bordered by a wall portion protruding substantially perpendicular from the window towards an interior of the housing, the wall portion configured to support the cartridge (e.g. the reservoir) within the housing without adhesion; the rear frame comprises one or more apertures configured to allow an airflow into the housing; and the housing is configured to hold the cartridge between the front and rear frames so as to allow the volatile composition to evaporate from the cartridge, said cartridges are held within the reusable housing without adhesion or attachment to the reusable housing and may be removed from the reusable housing without being touched by a user; and
either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

In such forms, the present disclosure provides a consumer product comprising a cartridge that is highly adapted to be used with a specific housing, where use of the cartridge with the housing specified in the instructions provides a number of advantages as discussed herein. These advantages may not be obtained if the cartridge is used with a housing different from that referred to in the instructions, or if a different cartridge is used with the housing. By using the cartridge with a housing as defined, a consumer will reduce plastics waste as compared to single-use volatile composition dispensers. The cartridges may be configured to be (a) held within a housing without adhesion or attachment, and/or (b) removable from the reusable housing without being touched by a user. This enables the present disclosure to provide a small, compact and portable cartridge having a high membrane evaporative surface area that is easy to remove from the housing at the end of its life. In particular, by not needing to touch the cartridge with their hands to remove it from a housing, a consumer is advantageously able to avoiding touching the membrane, which will be wetted with a volatile composition that typically includes a substantial amount of organic materials that are unpleasant to touch.

### BRIEF DESCRIPTION OF THE DRAWINGS

While the specification concludes with the claims particularly pointing out and distinctly claiming the examples, it is believed that the present disclosure will be better understood from the following description taken in conjunction with the accompanying drawings.

FIG. 1 is an illustration of a consumer product according to a configuration.

FIG. 2 is an illustration of a consumer product according to a configuration.

FIGS. 3A and 3B show front perspective views of a cartridge according to a configuration.

FIG. 4 shows a rear view of a cartridge according to a configuration.

FIGS. 5A to 5D show an example of a reusable housing that the cartridges disclosed herein may be used with.

FIG. 6 shows a cartridge according to a configuration.

FIG. 7 is an illustration of the effect of compressing a cartridge according to a configuration.

FIG. 8 shows an expanded view of a cartridge according to a configuration.

### DETAILED DESCRIPTION

Various configurations will now be described to provide an overall understanding of the principles of the structure, function, manufacture, and use of the apparatuses and methods disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure is defined solely by the claims. The features illustrated or described in connection with one example configuration may be combined with the features of other example configurations. Such modifications and variations are intended to be included within the scope of the present disclosure.

In one aspect, the present disclosure relates to a consumer product comprising:
(i) an outer packaging;
(ii) one or more cartridges containing a volatile composition; and
(iii) instructions for using the one or more cartridges with a reusable housing;
wherein the one or more cartridges are packaged within the outer packaging, and each comprise:
   a reservoir for interfacing with the reusable housing, the reservoir containing a volatile composition in liquid form and having an opening;
   a membrane enclosing the opening of the reservoir, the membrane configured to allow evaporation of the volatile composition; and
   a sealing substrate enclosing the opening of the reservoir, wherein:
the one or more cartridges are single-use cartridges for placing into the reusable housing. The consumer product, and in particular the outer packaging, one or more cartridges, and instructions, may have any of the additional features defined herein. The disclosure herein of embodiments that are narrower than this aspect should not be taken to limit the present disclosure to be narrower than this aspect of the present disclosure.

In some configurations, the consumer product may take the form of a refill pack of cartridges, where the refill package does not comprise the reusable housing that is referred to in the instructions. The components of the consumer product are discussed below.

### Outer Packaging

The outer packaging may be any appropriate packaging, such as a rigid or soft packaging. From the perspective of protecting the cartridges against accidental damage or rupture, the packaging may be rigid or semi-rigid. For example, the packaging may be formed from a plastics material, or from a cardboard material. In some configurations, the outer packaging may be formed from a combination of rigid and semi-rigid materials, such as a combination of plastics materials and cardboard materials.

In some configurations, the outer packaging may be formed from a majority of cardboard materials. For example, the outer packaging may be formed from at least 50 wt. % cardboard materials, such as at least 60 wt. %, at least 70 wt. %, at least 80 wt. %, at least 90 wt. %, at least 95 wt. % cardboard materials.

The outer packaging may take any appropriate form. In some configurations, the outer packaging may take the form of a blister or clam-shell packaging that may be made from a plastics material, a cardboard material, or a combination thereof. A blister packaging may have any appropriate construction, including a face seal blister, full-face seal blister, full card blister, and trapped blister. In some configurations, the outer packaging may take the form of a box, such as a cardboard box.

Suitable cardboard materials that may be utilized in the outer packaging include corrugated cardboard (such as single face, single wall, double wall, triple wall and honeycomb corrugated cardboard) and paperboard. In some configurations, the cardboard materials may be laminated with a plastics material.

Suitable plastics materials that may be utilized in the outer packaging include any transparent plastics material that is able to adequately protect the cartridges. Specific examples include PET (polyethylene terephthalate) and PVC (polyvinyl chloride), though a skilled person will appreciate that other plastics may be used.

### Cartridge

The one or more cartridges are packaged within the outer packaging to form a consumer product. For the sake of brevity, the one or more cartridges may be referred to herein as “the cartridge”.

The cartridge is a single-use disposable cartridge that contains a volatile composition for release to a surrounding environment, and once a cartridge is depleted of volatile composition it may be disposed of. The cartridge is for placing into a reusable housing, such that once the cartridge is depleted of volatile composition it may be removed from the housing and replaced by a new cartridge. The use of single-use cartridges with a reusable housing reduces the amount of material contributed to landfill as compared to products that are entirely single-use (i.e. where the housing is single-use), and also uses a lower volume of material (e.g. plastic) during the manufacturing process.

The cartridge comprises a reservoir, a membrane, and a sealing substrate. The reservoir contains the volatile composition and is enclosed by both the membrane and the sealing substrate, which may enclose the reservoir in any order. Thus, the membrane may enclose the sealing substrate, or the sealing substrate may enclose the membrane.

The cartridge may comprise an outer peripheral seal area, in which a peripheral portion of the reservoir and membrane may be sealed together. In configurations where the sealing substrate encloses the membrane, a peripheral portion of the sealing substrate may be sealed to a peripheral portion of the membrane at the outer peripheral seal area, i.e. a peripheral portion of each of the reservoir, membrane and sealing substrate may be sealed together at the outer peripheral seal area, provided that the sealing substrate may nevertheless be removed from the cartridge. This may be the case where the edges of the reservoir and membrane are coterminous. Alternatively, the edge of the reservoir may extend beyond the edge of the membrane, and the sealing substrate may be sealed directly to the reservoir at a peripheral region of the reservoir that is beyond the edge of the membrane. In configurations in which the membrane encloses the sealing substrate and the cartridge also comprises a rupture mechanism for rupturing the sealing substrate, the outer peripheral seal area may comprise only a peripheral portion of the reservoir and membrane. In such configurations, the cartridge may also comprise an inner peripheral seal area in which an inner peripheral portion of the reservoir is sealed to the sealing substrate. This may be achieved by providing the reservoir with an intermediate step at an inner peripheral portion (e.g. between the outer peripheral portion and a main body of the reservoir), where the sealing substrate may be sealed to the intermediate step of the reservoir.

The cartridge described herein advantageously allows a spent or finished cartridge to be removed from the housing without a user needing to touch the cartridge, thereby avoiding contact between a user's hands and organic components of the volatile composition throughout the entire cartridge life cycle. This may be achieved by providing the cartridge in a form that is configured to be placed into a reusable housing without adhesion or attachment to the reusable housing. For example, a housing may be opened and upended so that the cartridge falls out of the housing. This benefit of the examples of the present disclosure is particularly advantageous because a spent or finished cartridge will still contain a small amount of residual volatile composition, and so a membrane will be wetted with organic components even when the cartridge has reached the end of its effective life.

For example, the cartridge may be configured to be held within a housing without adhesion, and/or without attachment. In this context, “adhesion” refers to the use of an adhesive to secure the cartridge within the housing, for example an adhesive strip such as glue, adhesive tape or Velcro. Thus, in some configurations the cartridge does not comprise an adhesive on an external portion of the cartridge, which adhesive is intended to releasably bond the cartridge to a housing. “Attachment” refers to any mechanism of securing the cartridge within the housing that requires an active releasing or disengaging step and does not allow the cartridge to simply fall out of the housing when the housing is opened and upended. For example, “attachment” might refer to a hook or clamp, a part that is configured to be held securely within a slot in a housing and would require a manual removal step to be withdrawn from the slot, or any other attachment means for securing the cartridge to a housing and that would require a disengaging or releasing step.

In some configurations, the cartridge may be configured to be held within a housing by virtue of an attachment means that can be released or disengaged without touching the cartridge, such as via a button or slider.

In some configurations described herein, the cartridge is configured to be removed from a housing without being touched by a user. In this context, “without being touched by a user” is to be understood as enabling the removal of the cartridge without a user touching the cartridge, whether directly touching the cartridge, or indirectly touching the cartridge such as with the use of gloves or another implement such as a stick.

In some configurations, the cartridge may comprise one or more secondary reservoir portions. The secondary reservoir portions are arranged such that, when the cartridge is oriented for use, at least one secondary reservoir portion is gravitationally below the reservoir; and a total volume of the at least one secondary reservoir portion that is gravitationally below the primary reservoir portion is at least 4% of the initial volume of volatile composition.

In this context, “oriented for use” refers to the orientation that a cartridge might reasonably be placed in during use by a consumer, such as the intended configuration of a housing that the cartridge is intended to be placed in. For example, if a housing comprises a base, then oriented for use would refer to the orientation when the base is placed on a substantially flat surface. If a housing comprises a hook, then oriented for use would refer to the orientation resulting from the housing being suspended from said hook. “Oriented for use” may refer to the orientation that would result from following the instructions discussed herein.

As used herein, and provided the reservoir and the secondary reservoir portions are fluidly connected, when a secondary reservoir portion is “gravitationally below” the reservoir, then the secondary reservoir portion is positioned such that liquid present in the reservoir is able to flow under the force of gravity to the secondary reservoir portion. The liquid may flow through a connection (e.g. aperture) directly from the primary reservoir portion to the secondary reservoir portion, or via a conduit connecting the primary and secondary reservoir portions. When at least one secondary reservoir portion is gravitationally below the reservoir, a total volume of the at least one secondary reservoir portion that is gravitationally below the reservoir is at least 4% of the initial volume of volatile material. In the case where the secondary reservoir portion(s) include a part that is below the reservoir and a part that is not below the reservoir, the volume of the part that is below the primary reservoir portion is at least 4% of the initial volume of volatile material. Thus, when the cartridge according to the present disclosure contains less than 4% of the initial volume of volatile material and is oriented in this way, substantially all of the remaining volatile material is held in a secondary reservoir portion, and the reservoir is substantially empty. A person skilled in the art will appreciate that a small amount (e.g. droplets) of volatile material may remain in the reservoir. This advantageously informs a user that the cartridge is depleted of volatile composition and has reached the end of its useful life. This is beneficial because volatile compositions typically include a small amount of non-volatile material that will be present at end-of-life, and which may be visible in the reservoir. A user may become confused by this residual material in the reservoir, and erroneously believe that the cartridge is not depleted, leading to dissatisfaction with cartridge performance over a subsequent time period.

In some embodiments of the present disclosure, when the cartridge is oriented for use, a total volume of the at least one secondary reservoir portion that is gravitationally below the reservoir may be from 4% to 30% (e.g. from 5% to 20%, such as from 7% to 15%) of the initial volume of volatile material.

The reservoir, membrane, and sealing substrate are discussed in turn below.

### Reservoir

The reservoir contains the volatile composition, and has an opening that is enclosed by the membrane and sealing substrate.

The reservoir of the cartridge may typically be formed from a plastics material, which may advantageously be transparent to allow an easy view of a fill level of volatile composition within the reservoir. An example of a suitable material is polyethylene terephthalate (PET).

The reservoir is configured for interfacing with the reusable housing described herein. Thus, in some configurations the term “interfacing” may be understood as meaning that the reservoir is configured to be received by a window of a reusable housing, so that the cartridge is held securely within the housing when the housing is closed. The reservoir may therefore have a shape that is configured to correspond with a window of a reusable housing, so the reservoir portion may be received and fit snugly within the window.

In such configurations, it may be advantageous for the reservoir to be formed from a transparent material, so that the fill level of volatile composition within the reservoir is visible from outside the reusable housing, such as through the window.

The cartridge disclosed herein is a single-use cartridge for placing into a reusable housing. Thus, the cartridge typically does not comprise a housing of its own. In other words, the reservoir of the cartridge may be an outermost layer of the cartridge. In this context, “outermost” is to be understood as meaning that the cartridge do not include a substantial component outside the reservoir. For the avoidance of doubt, this does not exclude the presence of the membrane and sealing substrate enclosing an opening of the reservoir. In some configurations, the reservoir may nevertheless include a label or wrapping around the reservoir, which is intended to convey information to a user. However, the reservoir may be transparent as discussed herein, and in such cases the reservoir may typically not be covered by an additional label or wrapping so as to not obscure the reservoir.

The reservoir may have any appropriate shape. In order to hold the cartridge securely and prevent rotation of the cartridge, the reservoir may correspond to a shape of a window in a housing with which the cartridge is configured to be used. Thus, the reservoir may have an oval shape. The combination of a window and reservoir both having an oval shape ensures that the cartridge is held securely within the window and cannot rotate within the window. In addition, the absence of corners/vertices allow for the cartridge to be placed and removed from the window more easily than a polygonal shape which requires specific alignment. When an oval shaped cartridge is placed into an oval shaped window, the curved edges of the cartridge and window will naturally align the cartridge during insertion, improving case of use and user experience. The reservoir may have any appropriate size. For example, the reservoir may have a maximum dimension of from about 4.5 cm to about 5.5 cm. The reservoir may have an oval shape and an aspect ratio of from about 1.2 to about 1.8, such as about 1.3 to about 1.6. These dimensions may be measured at a maximum distance away from the membrane, in a plane that is parallel to the plane of the membrane. The reservoir may comprise a trapezoid-like taper as the reservoir extends away from the membrane (e.g. the reservoir may have a tapering cross-section with increasing distance from the membrane). This taper improves case of insertion into a window of a housing since the taper guides the cartridge into the window during insertion. The reservoir may have a variable depth, which may also increase case of insertion into a window of a housing as a less deep end of the reservoir may be inserted first, and a sloped section of the reservoir may help to align the reservoir within the window during insertion. Thus, the reservoir may have a depth at a first end that is greater than a depth at a second end. In some configurations, the reservoir may comprise a first end section, a second end section at an end opposing the first end section, and a sloped section therebetween. In some configurations, the sloped section may have a slope angle of from 2° to 35°, such as from 3° to 25°, such as from 4° to 15°. The first and second end sections may correspond to the respective end of the cartridge, such that the sloped section extends to and/or from the first and/or second end. Alternatively, the first and/or second end sections may have substantially uniform depth, with the sloped section therebetween. In this context a substantially uniform depth may correspond to a maximum to minimum depth ratio of from about 1 to about 1.4, preferably from about 1 to about 1.3, more preferably from about 1 to about 1.2, where the maximum and minimum depth are measured along a line bisecting the cartridge in a longitudinal direction.

It is worth noting that the depth of the reservoir can be useful, particularly where the scaling substrate is removable-removable sealing substrates are discussed in additional detail hereafter. For example, where the depth of the reservoir is sufficient, a user may grasp a sidewall of the reservoir while removing the sealing substrate. It is believed that a reservoir depth of at least 5 mm, more preferably at least 6 mm, even more preferably at least 7 mm, or most preferably at least 8 mm of depth can provide an adequate gripping surface when a user removes the scaling substrate. While the foregoing represents minimum depths of the reservoir which provide an adequate gripping surface, it is further believed that too high of a value for the reservoir depth can make handling the cartridge challenging. For example, it is believed that the reservoir depth can be 15 mm or less, preferably about 14 mm or less, more preferably about 13 mm or less or most preferably 12 mm or less. Of course, the minimum and maximum depths for the reservoir may be combined to create ranges of depth for the reservoir.

Additionally, as noted previously, where the depth of the reservoir varies along its length and/or width, it may be preferable to have the depth of the reservoir as described above adjacent a pull tab for the sealing substrate. This may improve the case of gripping the cartridge whilst removing the sealing substrate. For example, if the pull-tab is disposed on a side of the cartridge, then a maximum reservoir sidewall depth may be adjacent to the pull-tab, e.g. on the side of the cartridge.

As noted, the reservoir comprises a sidewall. A periphery may extend outboard of the sidewall. The periphery of the cartridge may comprise a sealing area where the membrane and scaling substrate are joined to the reservoir. Where the sealing substrate is removable, an adhesive dead area may be provided in the periphery. In order to provide sufficient adhesion of the membrane and sealing substrate, it is believed that the periphery should have a width (generally perpendicular to the sidewall) of at least 3 mm, more preferably at least 5 mm. In order to facilitate removal of the sealing substrate it is believed that the dead area may have a width of at least 1 mm or more preferably 2 mm, even more preferably at least 2.5 mm, from an outer edge of the periphery toward the sidewall. Greater than 2.5 mm of adhesive dead area is believed to detrimentally affect adhesion of the sealing substrate and membrane which may lead to leakage during shipping, shelving and/or during use.

### Membrane

The volatile composition is in liquid form and is configured to evaporate through a membrane. Accordingly, the cartridge comprises a microporous membrane, which for the sake of brevity may be referred to herein as “the membrane”. The membrane may enclose the reservoir such that volatile composition is unable to escape from the cartridge without passing through the membrane. The membrane may prevent the passage of liquid, such that the volatile composition is only able to escape the cartridge by evaporating through, or from, the membrane.

The membrane is vapor permeable and capable of wicking liquid, yet prevents free flow of liquid out of the membrane. Any suitable membrane may be used. Purely by way of example, certain properties that may result in advantageous membranes are discussed below. However, the present disclosure is not limited to membranes having the properties below, and any membrane known in the art that allows the volatile composition to evaporate may be used in the examples of the present disclosure.

The membrane may have any appropriate volume average pore diameter, such as from 0.01 μm to 0.5 μm, such as from 0.02 μm to 0.3 μm, such as from 0.05 μm to 0.2 μm, more particularly from 0.065 μm to 0.15 μm since this may provide improvements with regard to evaporation rate and controlling leakage or sweating of volatile composition. In certain configurations, the membrane may have a volume average pore diameter of from 0.065 μm to 0.15 μm, from 0.07 to 0.12 μm, from 0.07 to 0.11 μm, or 0.08 to 0.1 μm.

In some configurations, the membrane may have a pore size distribution such that at least 50%, such as at least 60%, such as at least 70%, such as at least 80% or such as at least 90% of the pores of the membrane have a pore diameter of from 0.065 μm to 0.15 μm.

The membrane may comprise (e.g. be formed from) any appropriate material, such as polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

The membrane may be formed from a single piece, or single sheet, of material. In other words, the membrane may not be laminated. Thus, the membrane may be formed from a single sheet of polyethylene having a thickness as described above.

Those of ordinary skill in the art will appreciate that the surface area of the membrane can vary depending on the user preferred size of the cartridge. In some configurations, the (evaporative) surface area of the membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

Particularly preferred membranes may have an evaporative surface area of from about 20 cm² to about 40 cm², such as from about 30 cm² to about 35 cm².

The membrane may form substantially all (e.g. at least 80%, at least 85%, at least 90% or at least 95%) of the surface area of a face of the cartridge. Thus, the cartridge may have a front face and a back face, and the membrane may form substantially all (e.g. at least 80%, at least 85%, at least 90% or at least 95%) of the surface area of the front or back face of the cartridge. In some configurations, the membrane may form substantially all (e.g. at least 80%, at least 85%, at least 90% or at least 95%) of a projection of the cartridge onto a plane, where the plane is selected to provide the maximum membrane area. This advantageously allows the membrane to have a maximised evaporative surface area for the size of the cartridge, leading to improved release of volatile composition.

Thus, in some configurations the membrane may have an evaporative surface area of from about 20 cm² to about 40 cm², such as from about 30 cm² to about 35 cm² and form substantially an entire face of the cartridge.

In some configurations, the membrane may have an evaporative surface area of from about 20 cm² to about 40 cm², such as from about 30 cm² to about 35 cm² and the cartridge may have a maximum dimension of less than 10 cm, preferably less than 9 cm. This advantageously means that the cartridge has a compact size whilst retaining a high evaporative surface area.

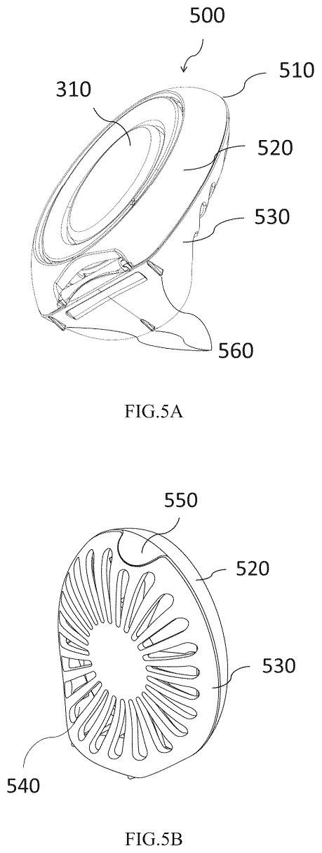

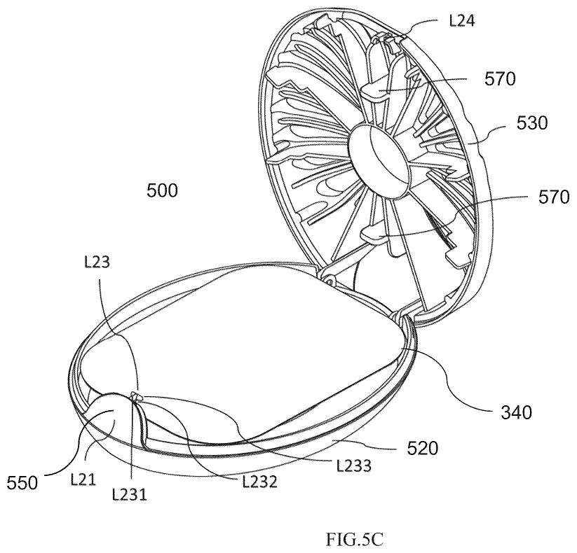

In such configurations, the membrane has a high size relative to the overall size of the cartridge. It is therefore particularly advantageous for the cartridge to be configured to be removed from a housing without being touched by a user, because it would otherwise be very difficult to avoid touching the membrane (which is wetted with volatile composition) when removing the cartridge from a housing. This may be achieved by ensuring that the cartridge is held within the housing without adhesion or attachment, so a user can simply upend an open housing to remove the cartridge, and does not need to disengage or unstick the cartridge from the housing. This benefit is illustrated by FIG. 5C, which shows how the cartridge may be positioned within a housing. When this housing is opened, it would be difficult to lift the cartridge out without touching the membrane, and so it is advantageous that the cartridge may instead be removed by simply upending the open housing and allowing the cartridge to fall out.

The membrane may have any appropriate porosity. For example, the membrane may have a porosity of from 45% to 70%, on a volume basis, such as from 45% to 65%. In certain configurations, the porosity may be from 50 to 70%, such as 55 to 65%.

The membrane may have any appropriate total pore volume, such as from 0.6 to 2 cm³/g. Typically, the total pore volume may be from 0.65 to 1.6 cm³/g, such as 0.7 to 1.5 cm³/g. In certain configurations, the total pore volume may be from 0.8 to 1.4 cm³/g.

The membrane may have any appropriate bulk density, such as from 0.3 to 0.8 g/cm³. Typically, the bulk density may be from 0.35 to 0.75 g/cm³, such as from 0.4 to 0.7 g/cm³. In certain configurations, the bulk density may be from 0.4 to 0.6 g/cm³.

Suitable membranes for the present disclosure include polyethylene membranes having the properties described herein, available from Microporous, LLC.

The membrane may comprise any suitable filler and plasticizer known in the art. Fillers may include finely divided silica, clays, zeolites, carbonates, charcoals, and mixtures thereof. In one configuration, the membrane may be filled with about 30% to about 80%, by total weight, of silica.

In one aspect of the present disclosure, the membrane may include a dye that is sensitive to the amount of volatile composition it is in contact with to indicate end-of-life. Alternatively, the membrane may change to transparent when in contact with a fragrance or volatile composition to indicate diffusion is occurring. Other means for indicating end-of-life that are known in the art are contemplated for the present disclosure.

The membranes described herein may advantageously provide a clear visual change when wetted with volatile composition, and when dry (whether before use or at end of life). Such visual changes may be more detectible when the membrane does not comprise a white pigment (e.g. TiO₂). Therefore, the membrane may comprise less than 5 wt. % of a white pigment, such as less than 1 wt. % of a white pigment, less than 0.1 wt. % of a white pigment, or less than 0.01 wt. % of a white pigment. The membrane may be free from a white pigment.

The visual change when the membrane is wetted as compared to dry may be more noticeable when the membrane comprises a coloured dye/pigment or a black dye/pigment. Therefore, the membrane may comprise a coloured or black dye/pigment, such as activated charcoal. Such a coloured or black pigment/dye (e.g. activated charcoal) may be present in any suitable amount, such as from 0.1 to 5 wt. %, e.g. 0.3 to 1 wt. %.

### Sealing Substrate

The cartridge comprises a sealing substrate that encloses the reservoir, and hence, encloses the volatile composition. This prevents evaporation of the volatile composition for as long as the sealing substrate is in place and intact. The sealing substrate may be removed or ruptured to allow the volatile composition to evaporate. This removal or rupturing may be referred to herein as “activating” or “activation of” the cartridge. The cartridge is configured to be activated before use, i.e. the sealing substrate is configured to be ruptured or removed before use. In this context, “use” refers to enabling the volatile composition to evaporate from the cartridge, i.e. perform its function of dispensing the volatile composition by evaporation. In other words, rupturing or removing the sealing substrate enables evaporation of the at least one liquid volatile composition from the cartridge.

Non-limiting examples of suitable sealing substrates include an impermeable film, foil, or laminate, such as a flexible (e.g. polymeric) film, a flexible (e.g. metal) foil, or a composite material (e.g. a foil/polymeric film laminate). The impermeable film, foil or laminate is provided adhered to the cartridge to prevent evaporation of volatile composition. A particular example of a suitable scaling substrate is aluminium foil.

The sealing substrate may have any appropriate thickness, such as from 10 μm to 1 mm, from 15 μm to 100 μm, from 18 μm to 50 μm, or from 20 μm to 35 μm.

As mentioned above, there are two possible configurations for the order of the scaling substrate and the membrane.

In some configurations, the sealing substrate may be positioned between the membrane and the volatile composition, preventing the volatile composition from contacting the membrane. Thus, the cartridge may be configured such that an external force applied to the cartridge may cause rupture of the scaling substrate and allow volatile composition to pass through the ruptured scaling substrate and come into contact with the membrane. The volatile composition may then impregnate the membrane, from which it may evaporate. The cartridge may comprise a rupture mechanism, and rupture of the sealing substrate may be caused by actuation of the rupture mechanism, which may be achieved by a force applied to the cartridge at an appropriate location. Suitable rupture mechanisms are described in detail in U.S. Pat. Nos. 10,561,754, 10,561,755 and 10,561,756. The rupture mechanism may be actuated by a force applied by a housing with which the cartridge is configured to be used, such as a housing that comprises rib elements configured to actuate the rupture mechanism when the cartridge is placed inside the housing. Alternatively, a force applied to the cartridge may cause rupture of the sealing substrate directly, by applying pressure to the scaling substrate through the membrane. Thus, the membrane may be capable of deformation such that as a force is applied to the membrane (e.g. a force applied by rib elements present on a housing as described herein), the membrane is able to deform in the direction of the applied force, towards the scaling substrate. In such configurations, the sealing substrate may be formed from a material that is substantially inelastic, such as a metal foil. When the aforementioned force is applied to the membrane, the membrane deforms and comes into contact with the sealing substrate. The scaling substrate is unable to sufficiently deform or flex in the direction of the force, and so may be ruptured by the force. In such configurations, it is important that as the membrane is deformed by the applied force and presses against the sealing substrate, the sealing substrate ruptures before the membrane is damaged. In other words, the applied force causes the sealing substrate to be ruptured, whilst the membrane is deformed without breaking, rupturing or tearing. Thus, in some configurations, the membrane may enclose the sealing substrate, and the sealing substrate may be configured to be ruptured by a force applied to the sealing substrate through the membrane. In other configurations, the membrane may be positioned between the volatile composition and the sealing substrate, such that the volatile composition is in contact with the membrane but cannot evaporate and leave the cartridge because the membrane is enclosed by the sealing substrate. In such configurations, the cartridge may also be configured for use with a housing that, like the foregoing, comprises ribs which directly rupture the sealing substrate. However, this may result in only small holes in the sealing substrate, limiting the rate at which the volatile composition may evaporate from the cartridge. Therefore, in such configurations, the sealing substrate may be provided as a removable component (such as a removable film, foil or laminate) that encloses the membrane and prevents evaporation of the volatile composition before the sealing substrate is removed. When a scaling substrate of this type is removed from the cartridge, the entire membrane may be exposed to a surrounding environment, enabling an increased evaporation rate of volatile composition. In such cases, the scaling substrate may be provided with a pull tab to allow for easy removal of the scaling substrate from cartridge prior to use by a consumer.

Where the sealing substrate is provided with a pull tab to facilitate removal of the scaling substrate, the pull tab may be any suitable size. For example, the pull tab may have a length of from about 20 mm or less, preferably about 15 mm or less, or more preferably about 10 mm or less. As another example, the pull tab may have a length of from about 2 mm to about 20 mm, preferably from about 5 mm to about 15 mm, or more preferably from about 5 mm to about 10 mm, specifically including all values within these ranges and any ranges created thereby. Similarly, the width of the pull tab may be about 20 mm or less, preferably about 15 mm or less or more preferably about 10 mm or less. As another example, the pull tab may have a width of from about 2 mm to about 20 mm, preferably from about 5 mm to about 15 mm, or more preferably from about 5 mm to about 10 mm, specifically including all values within these ranges and any ranges created thereby. The pull tab may be positioned in any suitable location on the scaling substrate. Preferably the pull tab is disposed adjacent the seal area.

Where the length and/or width of the pull tab exceed the above, the cartridges may be difficult to package. For example, the housing and the cartridge may be packaged together or separately. Where the cartridge is disposed within the housing, the housing may be in the open position or the closed position. When in the closed position, it would be desirable to ensure the pull tab is similarly disposed within the housing as well. Where the cartridge is disposed outside of the housing, it may be useful to show a portion of the pull tab to a consumer to demonstrate the facility with which the cartridges may be activated; however, where the length and/or width exceed the above values, it may be difficult to show only a portion while hiding the remainder of the pull tab.

As described, in some configurations, the sealing substrate may be a rupturable substrate. In other configurations, the sealing substrate may be a removable sealing substrate.

When the sealing substrate is ruptured during closing of a housing as described herein, a membrane located externally from the sealing substrate may become wetted with the volatile composition. Since the volatile composition typically comprises various organic compounds and solvents, users typically prefer to avoid needing to touch a wetted membrane. The use of a rupture mechanism described herein advantageously allows the cartridge to be activated without a user needing to touch the cartridge during the activation step, thereby avoiding any contact between a user's hands and organic components of the volatile composition. Therefore, in some configurations described herein, the sealing substrate is configured to be ruptured during closing of a reusable housing.

### Volatile Composition Dispenser

The combination of a cartridge and a reusable housing may be referred to as a volatile composition dispenser. Thus, placing one of the one or more cartridges into a reusable housing may typically form a volatile composition dispenser, which may be used to release the volatile composition over time.

Therefore, the cartridge described herein may be placed in a reusable housing to form a volatile composition dispenser. For example, a volatile composition dispenser may comprise (i) a reusable housing; and (ii) a cartridge as described herein held within the housing. The cartridge is typically held within the housing without adhesion or attachment to the housing, as is described in more detail below.

In this way, the cartridge can be used to dispense at least one volatile composition and/or other solution or composition, such as a perfume, a fragrance, and/or an insecticide, for example, to a surrounding area or atmosphere. The volatile composition can comprise a single chemical or a single material that is capable of entering the vapor phase under atmospheric conditions or, more commonly, the volatile composition can comprise a mixture of chemicals and/or materials that are capable of entering the vapor phase under atmospheric conditions.

The cartridge may be intended to be used within an interior space, such as an interior space in a building or a passenger compartment of a vehicle, although the present disclosure is not limited to such use and those of skill in the art will understand that the dispenser can be configured for use in any appropriate environment, and can be configured to dispense any suitable solution, chemical, material, and/or composition.

The cartridge may be configured to dispense a volatile composition in a continuous manner without requiring any energy input, i.e. the cartridge (and corresponding housing) may be non-energized. “Non-energized” can mean that the apparatus is passive and does not require to be powered by a source of external energy. The cartridge and any associated housing does not need to be powered by a source of heat, gas, or electrical current, and the volatile composition is generally not delivered by aerosol means (e.g. the cartridge may not include components under an elevated pressure).

The continuous emission of the at least one volatile composition can be for any suitable length, such as up to 20 days, 30 days, 40 days, 60 days, 90 days, shorter or longer periods, or any period between 10 to 90 days, for example. Of course, composition having greater or lesser volatility may be provided in the cartridge to increase or decrease its useful life. Also, the cartridge's useful life may be dependent on the conditions (i.e., temperature, pressure, moisture content, airflow etc.) in which it operates.

### Instructions for Use

As mentioned herein, the consumer product comprises instructions for using the one or more cartridges with a reusable housing. In this way, the instructions define a housing with which the cartridges are to be used. In this context, using the cartridges may be understood as placing the cartridge into the reusable housing that is defined in the instructions, in such a way that the reusable housing supports the cartridge and enables an effective release of volatile material without causing spillage or leakage of the volatile material. The use of the cartridge with a housing as specified in the instructions may also provide additional advantages discussed herein, which advantages may not be obtained if the cartridge is used with a housing that is not as specified in the instructions. These advantages are discussed in detail herein with respect to the housing.

While the instructions may be provided within the outer packaging, e.g. as an insert within the outer packaging, or as a marking on the one or more cartridges themselves, it is not necessary for the instructions to be physically packaged within the outer packaging. The instructions may be provided in any format such that they are provided to the consumer at the point or purchase or use of the consumer product. Thus, it is explicitly contemplated herein that the instructions may be present on the exterior of the outer packaging, alongside the outer packaging as a separate component (e.g. on a shelf alongside the consumer product) or provided electronically during use or purchase (e.g. as part of an advertisement or sales listing accessed electronically).

The instructions for use instruct a user to use the one or more cartridges with a reusable housing, for which the cartridges are adapted. The instructions may take the form of a written note or graphical indication that the one or more cartridges are suitable for, or compatible with, a specific reusable housing. Thus, the instructions may direct a user to place the one or more cartridges into a housing that has features defined hereinbelow. Therefore, in some configurations the present disclosure provides a consumer product wherein the instructions direct a user to use the one or more cartridges with a reusable housing that has any combination of features defined herein.

The instructions may direct a user to place the one or more cartridges into a single specific reusable housing, such as a specific branded housing, where such a housing has the properties defined in the appended claims. Alternatively, the instructions may provide a list of possible reusable housings that the one or more cartridges may be used with, where each of the listed housings have the properties defined in the appended claims.

### Reusable Housing

As noted, the cartridge is a single-use cartridge for placing into a reusable housing. For brevity, the reusable housing may be referred to herein simply as “the housing” or “a housing”.

The instructions may direct a user to place a cartridge as defined herein into a specific reusable housing. The reusable housing that may be referenced by the instructions is discussed below.

The housing may be a non-energized housing. Suitable housings may have a front frame and an opposing rear frame. The front frame and the rear frame may be movable with respect to one another allowing the housing to express a closed position and an open position. The front frame and the rear frame may be pivotally connected (e.g. via a hinge) to one another in a clam-shell arrangement, or may be completely separable.

In order to advantageously improve case of use, the housing may comprise a hinge as described herein (e.g. the housing may comprise a front and rear frame that are pivotally connected). The hinge/pivotal connection enables a user to make use of the lever effect when closing the housing and applying a compressive force as described herein.

Regardless of the connection between the front frame and the rear frame, the front frame and/or rear frame may comprise separation tabs and/or separation slots which can facilitate grasping by a user of the front frame and/or rear frame and also facilitate separation of the front frame from the rear frame. Where separation tabs are provided on both the front frame and the rear frame, the separation tabs may be spaced from one another to facilitate grasping by a user. For example, one or more separation slots may be included at the interface between the front frame and the rear frame or may be comprised wholly by the front frame or rear frame. Where more than one separation slot is utilized, a first separation slot may be positioned on one side of the housing and a second separation slot may be positioned on an opposite side of the housing.

Where separation tabs are utilized, the front frame may comprise one or more front separation tabs that are spaced from one another, and the rear frame may similarly comprise one or more rear separation tabs that are spaced from one another. In such configurations, the one or more front separation tabs may similarly be spaced from the one or more rear separation tabs.

The housing may typically be formed from a plastics material, such as a polypropylene. A person skilled in the art will be aware of other suitable plastics that may be used.

The front frame and rear frame may together comprise a locking structure to releasably interlock the front and rear frames together upon closing of the housing. Thus, the locking structure may releasably lock the housing in a closed state. The locking structure may be disposed at a movable end (e.g. the second end) of the housing, which movable/second end is opposing to a first end in which the front and rear frames may be connected, e.g. via a hinge. Various locking structures can be utilized, including, for example, latches, snap fits, clasps and catches. Representative latch locking structures include through-latch and button latch structures.

The locking structure may be configured to produce a sound of at least 50 dB (such as from 50 dB to 65 dB) upon closing the housing and engaging the locking structure. This sound confirms to a user that the housing has been properly closed and is locked in the closed state. This sound is particularly advantageous when the housing is used with a cartridge that is activated by closing the housing, as is described herein, because the sound confirms to a user that the cartridge has been activated. This overcomes a problem with prior art volatile composition dispensers involving button activation mechanisms where a user was often unsure if a press of the button had properly activated a cartridge. The sound may also increase user satisfaction. The sound may be caused, for example, as the deformable overhang button portion reverts from a deformed state to a native state and/or as a latch engages with an intended corresponding structure.

As mentioned herein, the housing (e.g. a rear frame) may comprise one or more rib elements, which may be configured to apply a compressive force on the cartridge, for example by impacting the cartridge when the housing is in a closed state. Typically, such rib elements are configured to apply the compressive force on the membrane of a cartridge when the housing is in a closed state (whether through direct impact or through one or more additional components or layers).

The rib elements may take the form of one or more protrusions extending from the rear frame towards the middle of the housing. The rib elements may have any appropriate size and shape, such as a cubic, cuboidal, cylindrical, conical or polygonal shape that may have straight or curved edges and faces. The rib elements may have any appropriate size, but may typically have a maximum dimension extending from the rear frame of about 0.1 to 1 cm, and a maximum dimension parallel to the plane of the rear frame of about 0.1 to 2 cm. However, a person skilled in the art will appreciate that larger or smaller rib elements may be used.

In some configurations in which the rib elements are configured to apply a compressive force directly on the membrane during use, the rib elements should be of an appropriate size and shape that does not rupture, pierce, or otherwise damage the membrane.

The rib elements may comprise a cartridge-contacting surface that is substantially flat or rounded.

The housing (e.g. a front frame) may comprise a window. The window advantageously allows for the cartridge to be visible from outside the housing, and this may allow for a fill level of volatile composition to be easily determined without opening the housing. The window may be a cut-out portion of the housing (e.g. of the front frame), or the window may be a part of the housing (e.g. of the front frame) that is made from a transparent material. In some configurations, the window may be configured to receive a part of the cartridge, such as the reservoir. This helps to securely hold the cartridge in place when the housing is closed, without requiring any adhesive or other adhering means. In some such configurations, the window may be a cut-out portion of the front frame, which is configured to receive a part of the cartridge (e.g. the reservoir).

The window portion may have any appropriate shape. In order to hold the cartridge securely and prevent rotation of the cartridge, the window may have an oval shape. The window may have any appropriate size. For example, the window may have a maximum dimension of from about 4.5 cm to about 5.5 cm. The window may have an oval shape and an aspect ratio of from about 1.2 to about 1.8, such as about 1.3 to about 1.6.

In configurations where the housing (e.g. a front frame) comprises a window that is configured to receive a part of the cartridge (such as the reservoir), then at least a part of a perimeter of the window may be bordered by a wall portion protruding substantially perpendicular from the window towards an interior of the housing. The wall portion may be configured to support the cartridge within the housing without adhesion. For example, a part of the cartridge may rest on the wall portion when the cartridge is held within the housing. This allows a cartridge to be supported and securely held within the housing, but quickly and easily removed by a user because the cartridge is not adhered to the housing, as is discussed in more detail herein.

The wall portion may have any appropriate shape that allows the wall portion to support a cartridge within the housing without adhesion. For example, the wall portion may comprise a solid (e.g. uninterrupted) wall, or may alternatively comprise interrupted sections of wall, or may be formed from a plurality of protrusions. A person skilled in the art will appreciate that any of these configurations may be utilized to support a cartridge within the housing.

To facilitate emission of the volatile composition, the housing (e.g. a rear frame) may comprise one or more apertures which allow evaporated volatile composition to exit the housing. The apertures may be configured as described in U.S. Pat. No. 11,207,440. Additionally, the housing (e.g. a front frame) may similarly comprise one or more apertures which facilitate airflow through the housing. In some configurations, the aperture comprised by the front frame may have a lower aperture area than that of the one or more apertures in the rear frame.

A peak force required to close the housing when the housing contains the cartridge as defined herein, including engaging the locking structure when present may be from 20 N to 120 N, such as from 45 N to 110 N, from 50 N to 105 N, or from 55 N to 100 N, for at least 10 locking cycles. The higher peak force required to close the housing when it contains a cartridge may be caused by the force that is required to rupture the sealing substrate and/or compress of the cartridge, in addition to the force required to activate the locking mechanism.

The peak force to close the housing may be measured by securing the front frame at an angle such that, when the housing is closed, a seam between the front and rear frames is parallel to the horizontal. The rear frame may then be placed in an equilibrium position proximate to the front frame, e.g. where components of a locking structure are in contact but the locking structure is not engaged. The peak force may then be measured by applying a force on a dimensional centre point of the rear frame of the housing in a vertically downwards direction, and determining the peak force required to close the housing.

As mentioned above, the cartridge may be configured to be removed from the housing without being touched by a user. Therefore, in some configurations, the housing is configured such that when the housing is in an open configuration, a cartridge placed within the housing may be removed by upending the housing.

Thus, in some configurations, the housing may be an openable housing (e.g. having a clam-shell structure), and the housing and cartridge may be configured such that when a cartridge located within the housing has reached end of life, the cartridge may be removed from the housing by the steps:
(i) opening the housing; and
(ii) upending the housing,
without a step of disengaging or disadhering the cartridge from the housing.

As mentioned herein, the instructions of the consumer product may direct a consumer to place the cartridge into a housing as defined herein. Specific examples of housings that may be referred to by the instructions include I and II listed below. For the avoidance of doubt, these housings may include any additional features discussed herein.

I. A housing comprising a front frame and a rear frame that are capable of moving away from or toward each other for opening and closing the housing, wherein:
the front frame comprises a window configured to receive a part of the cartridge (e.g. the reservoir);
at least a part of a perimeter of the window is bordered by a wall portion protruding substantially perpendicular from the window towards an interior of the housing, the wall portion configured to support the cartridge (e.g. the reservoir) within the housing without adhesion;
the rear frame comprises one or more apertures configured to allow an airflow into the housing; and
the housing is configured to hold the cartridge between the front and rear frames so as to allow the volatile composition to evaporate from the cartridge.

II. A housing comprising a front frame and a rear frame that are capable of moving away from or toward each other for opening and closing the housing, wherein the rear frame comprises one or more rib elements that are configured to apply a compressive force on the cartridge when the housing is in a closed state and the cartridge is positioned within the housing. In such cases, the compressive force may cause a reduction in a thickness of the reservoir of the cartridge when held within the housing, as compared to a thickness of the reservoir before being subjected to the compressive force.

### Compressive Force

As described herein, a reusable housing may comprise one or more rib elements (e.g. as part of a rear frame), which rib elements may be configured to apply a compressive force on the cartridge when the cartridge is located within the reusable housing and the housing is closed. An opposing side of the cartridge may be held in place by the housing (e.g. a front frame), such that the compressive force is generated by the compression of the cartridge within the housing (e.g. between the front frame and the rib elements). While the compressive force may typically be generated by compression between a front frame and rib elements, for the sake of brevity and simplicity, the compressive force is described herein in relation to the action of the rib elements on the cartridge.

Typically, the rib elements may be configured to apply the compressive force on the membrane of a cartridge when the housing is in a closed state, either through direct impact of the rib elements on the membrane, or through one or more additional components or layers. The compressive force may be applied to the cartridge for substantially the entire lifetime of the cartridge while it is positioned within the housing. In other words, the compressive force may be applied to the cartridge for substantially as long as the cartridge is present within the housing and volatile composition is evaporating from the cartridge. In this context, the term substantially may mean at least 80% of the time, at least 85% of the time, at least 90% of the time, or at least 95% of the time.

As used herein, a compressive force refers to a force that acts to compress a part of the cartridge, and may be understood as a force that acts on a surface of the cartridge, in a direction towards an interior of the cartridge. The compressive force may act substantially perpendicular to a surface of the cartridge, but a person skilled in the art will understand that the compressive force may act at an angle relative to a surface of the cartridge and still provide the benefits discussed below. The compressive force may typically act at a direction that is substantially perpendicular to a surface of the cartridge, such as from 60° to 120° to a surface of the cartridge.

The compressive force may have any appropriate magnitude. The magnitude of the compressive force should be high enough that the compressive force is able to achieve the effects described below, but should not be excessively high such that it undesirably ruptures or otherwise damages the cartridge. Thus, the magnitude of the compressive force may be, for example, from about 30 N to about 120 N, such as from about 35 N to about 100 N, such as from about 40 N to about 90 N. A person skilled in the art will appreciate that smaller or greater compressive forces may be suitable in some circumstances.

The compressive force may be approximated as equal to the opening force described below, and for the purposes of the present disclosure, the magnitude of the compressive force may be measured by measuring the opening force as described herein.

This compressive force provides several benefits that are described below. Typically, these benefits apply when a cartridge as described herein is reversibly/elastically compressed or deformed by the compressive force. For example, the cartridge may be compressible or deformable, and typically the membrane and/or reservoir of the cartridge may be compressible or deformable. In this context, “compressible or deformable” refers to at least partially reversible compression and deformation, such that when removed from the housing, a cartridge at least partially reverts towards its original shape.

First, the compressive force applied by the rib elements on the cartridge causes an equal and opposite reaction force applied by the cartridge on the housing, which reaction force biases opening of the housing. This reaction force may be referred to herein as an “opening force”. The opening force may have a magnitude that is equal to the magnitude of the compressive force described above. In some configurations, the opening force may have a magnitude of from about 3 N to about 140 N, from about 10 N to about 100 N, from about 20 N to about 60 N, preferably from about 25 N to about 45 N. The opening force may be measured by closing the housing with a new cartridge, securing the front or rear frame at an angle such that a seam between the front and rear frames is parallel to the horizontal and measuring the peak vertical opening force at a midpoint of the housing when the locking structure is released/disengaged (taking the force of gravity into account).

Typically, the housing comprises a locking structure for releasably locking the housing in a closed state, such that the opening force does not cause the housing to open while the locking structure is engaged. However, when the locking structure is released or disengaged, the housing will at least partially spring open due to the opening force applied on the housing by the cartridge held within the housing. Thus, in some configurations described herein, when the housing is in a closed state, the cartridge applies an opening force on the housing, where the term “opening force” is to be understood as a force that biases opening of the housing. This advantageously increases the case of opening the housing, because when the locking structure is disengaged or unlocked, the housing will at least partially spring open, allowing for an easy grip of the front and rear frames.

Second, the cartridge includes a reservoir containing a volatile composition. When the compressive force acts on a deformable part of the reservoir, or the membrane enclosing the reservoir, a resulting deformation may decrease the thickness of the reservoir in the direction the force acts along. Typically, the compressive force acts in a direction that is substantially perpendicular to a plane of the membrane, such as from 60° to 120° to the membrane. When this direction is a horizontal direction, which may be the case when an appropriate housing is in an intended upright configuration as shown in FIG. 5B, the reduced horizontal thickness of the reservoir causes an increase in a height of the liquid volatile composition within the reservoir. As shown in FIG. 7, this provides the appearance of a higher fill level, increasing consumer satisfaction with the volatile composition dispenser, especially when a fill level of volatile composition is easily visible through a window of the front frame of the housing.

Third, when the compressive force acts on a deformable part of the reservoir, or the membrane enclosing the reservoir, the resulting deformation ensures that the cartridge is held tightly within the housing and does not move or rattle within the housing when the housing is moved. This provides a premium feel to the product, increasing user satisfaction. The effect of this benefit may be increased when the front frame of the housing comprises a window configured to receive the reservoir of the cartridge, as described herein.

### Volatile Composition

The volatile composition can comprise, but is not limited to, a substance that can function as an air freshener, a deodorant, an odor neutralizing material, an odor blocking material, a malodor counteractant, an odor masking material, an aromatherapy material, an aromachology material, an insecticide, air and/or surface sanitizer, and/or a combination thereof. In other various configurations, the volatile composition can comprise other various materials that can act in their vapor phase to modify, enhance, and/or treat an atmosphere or an area outside of the cartridge.

The term “volatile composition” as used herein, refers to a material that is vaporizable at room temperature and atmospheric pressure without the need of an energy source. The volatile composition may be a composition comprised entirely of a single volatile material. The volatile composition may also be a composition comprised entirely of a volatile material mixture (i.e. the mixture has more than one volatile component). Further, it is not necessary for all of the component materials of the composition to be volatile. Any suitable volatile composition in any amount or form, including a liquid or emulsion, may be used.

Liquid suitable for use herein may, thus, also have non-volatile components, such as carrier materials (e.g., water, solvents, etc). It should also be understood that when the liquid is described herein as being “delivered”, “emitted”, or “released,” this refers to the volatilization of the volatile component thereof, and does not require that the non-volatile components thereof be emitted.

The volatile composition can be in the form of perfume oil. Most conventional fragrance materials are volatile essential oils. The volatile composition can be a volatile organic compound commonly available from perfumery suppliers. Furthermore, the volatile composition can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like. In the case of air freshener or fragrances, the different volatile materials can be similar, related, complementary, or contrasting.

It may be desirable for the volatile composition to be in the form of a liquid at 25° C. As explained herein, the microporous membranes used in the present disclosure may have advantageously increased visual appearance changes when wetted with volatile composition.

The volatile composition may have a combined vapour pressure of at least 8 Pa at 25° C., such as at least 30 Pa at 25° C.

In addition to, or as part of, the volatile composition, the cartridge may include any known malodor composition to neutralize odors. Suitable malodor compositions include cyclodextrin, reactive aldehydes and ionones. In particular, the volatile composition may comprise a volatile carbonyl containing compound having a vapor pressure of at least 0.025 torr at 25 degrees Celsius, wherein the volatile carbonyl containing compound is selected from the group consisting of: volatile aldehydes, ketones, and mixtures thereof.

While not wishing to be bound by theory, the continuous delivery of a volatile composition may be a function of various factors including membrane pore size; membrane surface area; the physical properties of a volatile composition, such as molecular weight and saturation vapor pressure (“VP”); and the viscosity and/or surface tension of the volatile composition.

The volatile composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. In one embodiment, the volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

Two exemplary volatile compositions comprising a volatile material mixture having volatile materials of varying VPs are set forth below in Tables 1 and 2. These compositions are shown by way of illustration and are not intended to be in any way limiting of the examples of the present disclosure.

TABLE 1

| Wt % | Low VP (torr) | High VP (torr) |
|---|---|---|
| 27.71 | 0.175 | 0.325 |
| 20.78 | 0.0875 | 0.1125 |
| 13.86 | 0.0625 | 0.0875 |
| 8.66 | 0.0375 | 0.0625 |
| 8.66 | 0.0175 | 0.0325 |
| 6.93 | 0.00875 | 0.01125 |
| 6.93 | 0.00625 | 0.00875 |
| 3.18 | 0.00375 | 0.00625 |
| 1.27 | 0.00175 | 0.00325 |
| 0.95 | 0.000875 | 0.001125 |
| 0.64 | 0.000625 | 0.000875 |
| 0.32 | 0.000375 | 0.000625 |
| 0.09 | 0.000175 | 0.000325 |

TABLE 2

| Wt % | Low VP (torr) | High VP (torr) |
|---|---|---|
| 33.38 | 0.175 | 0.325 |
| 25.75 | 0.0875 | 0.1126 |
| 19.07 | 0.0625 | 0.0875 |
| 13.86 | 0.0375 | 0.0625 |
| 4.00 | 0.0175 | 0.0325 |
| 1.50 | 0.00875 | 0.01125 |
| 0.50 | 0.00625 | 0.00875 |
| 0.72 | 0.00375 | 0.00625 |
| 0.55 | 0.00175 | 0.00325 |
| 0.27 | 0.000875 | 0.001125 |
| 0.20 | 0.000625 | 0.000875 |
| 0.13 | 0.000375 | 0.000625 |
| 0.07 | 0.000175 | 0.000325 |

The viscosity of a volatile composition may control how and when a volatile composition is delivered to the microporous membrane. For example, less viscous compositions may flow faster than the more viscous volatile compositions. Thus, the membrane may be first wetted with the less viscous materials. To help prevent liquid from seeping through the microporous membrane, volatile compositions may have viscosities less than about 23 cP and surface tension less than about 33 mN/m.

In one embodiment, the volatile composition may have a viscosity of about 1.0 cP to less than about 25 cP, alternatively about 1.0 cP to less than about 23, alternatively about 1.0 cP to less than about 15 cP.

The volatile composition may be designed such that the composition may include a surface tension of about 19 mN/m to less than about 33 mN/m, alternatively about 19 mN/m to less than about 30 mN/m, alternatively about 19 mN/m to less than about 27 mN/m.

In addition to the configurations discussed above, the present disclosure also provides a consumer product that does not contain instructions for use. Such a consumer product may contain cartridges that are configured to achieve the benefits described herein when placed into a housing having the features described herein. For example, the cartridges may be for engaging with the housing, operably engageable with the housing and/or capable of engaging with the reusable housing described herein. Therefore, in some configurations, the present disclosure provides a consumer product comprising:
(i) an outer packaging; and
(ii) one or more cartridges containing a volatile composition, the one or more cartridges being single-use cartridges for placing into a reusable housing and packaged within the outer packaging, and each comprising:
   a reservoir for interfacing with the reusable housing, the reservoir containing a volatile composition in liquid form and having an opening;
   a membrane enclosing the opening of the reservoir, the membrane configured to allow evaporation of the volatile composition; and
   a sealing substrate enclosing the opening of the reservoir, wherein:
the one or more cartridges are configured such that, when placed into a housing described herein, said cartridges are held within the reusable housing without adhesion or attachment to the reusable housing and may be removed from the reusable housing without being touched by a user; and either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

Various configurations will now be described to provide an overall understanding of the principles of the structure, function, manufacture, and use of the apparatuses and methods disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure are defined solely by the claims. The features illustrated or described in connection with one example configuration may be combined with the features of other example configurations. Such modifications and variations are intended to be included within the scope of the present disclosure.

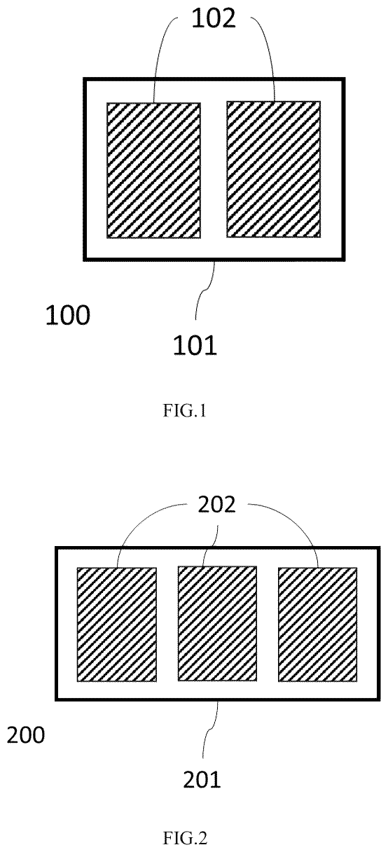

FIG. 1 shows a consumer product 100 comprising an outer packaging 101 and two cartridges 102. Instructions may be provided electronically, or inside, outside, or alongside the consumer product 100.

FIG. 2 shows a consumer product 200 comprising an outer packaging 201 and three cartridges 202. Instructions may be provided electronically, or inside, outside, or alongside the consumer product 200.

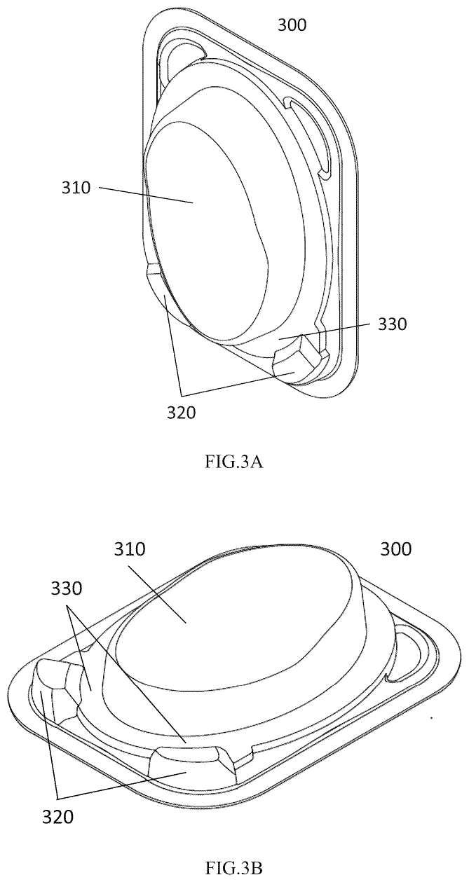

FIGS. 3A and 3B show two views of a cartridge 300. The cartridge 300 comprises a reservoir 310. The membrane and scaling substrate are not visible from the views shown in FIGS. 3A and 3B. The cartridge 300 also comprises two secondary reservoir portions 320. The reservoir 310 contains a volatile material, which volatile material is configured to evaporate from the reservoir 310 after an activation step is performed, i.e. after the sealing substrate is ruptured or removed. The cartridge 300 of FIGS. 3A and 3B is depicted with a secondary reservoir portion 320 which are arranged gravitationally below the reservoir 310 when the cartridge 300 is oriented for use. A conduit region 330 surrounding the reservoir 310 acts as a conduit to fluidly connect the reservoir 310 to the secondary reservoir portions 320. The reservoir 310 and secondary reservoir portions 320 are enclosed by a microporous membrane (not shown) located on the reverse side of the cartridge 300. The microporous membrane is configured to allow evaporation of volatile material contacting the membrane. While the cartridge 300 shown in FIGS. 3A and 3B contains secondary reservoir portions 320, a skilled person will appreciate that the examples of the present disclosure does not require that secondary reservoir portions are present in the cartridges disclosed herein.

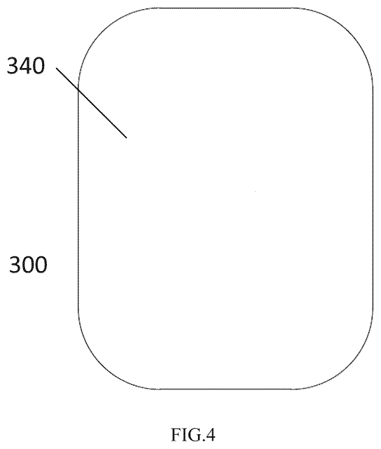

FIG. 4 shows a rear view of a cartridge 300 according to the present disclosure. The cartridge 300 comprises a scaling substrate 340 that encloses the membrane and the reservoir (not shown). The membrane is configured to allow evaporation of volatile material once the scaling substrate 340 is removed from the cartridge. In the cartridge 300 shown in FIG. 4, the entire back surface of the cartridge 300 is composed of the sealing substrate 340, though a person skilled in the art will appreciate that this is not a requirement of the cartridges disclosed herein.

FIG. 5A shows an example apparatus 500 for delivering a volatile composition (e.g. a volatile composition dispenser). The apparatus 500 comprises a reusable housing 510 having front frame 520, and a rear frame 530. The apparatus further comprises a cartridge as disclosed herein located within the reusable housing 510. The reservoir 310 of the cartridge is visible through the front frame 520 of the reusable housing 510, and is depicted being received by a window of the reusable housing. The rear frame 530 comprises feet 560 to provide a stable base when the apparatus 500 is placed on a surface. In FIG. 5A, other parts of the cartridge are not visible through the front part 520 of the reusable housing 510.

FIG. 5B shows a more complete view of the back part 530 of the reusable housing 510, which comprises a plurality of apertures 540 for exposing an evaporative portion (e.g. a membrane) of the cartridge. The reusable housing 510 is openable so that the cartridge may be replaced once it has reached end of life, and the back part 530 also comprises a locking structure 550 (partially shown) for releasably closing the reusable housing.

FIG. 5C shows the apparatus 500 in an open configuration, and shows a cartridge lying in the housing, with the membrane 340 visible and the reservoir (not shown) received by the window of the front frame 520. The locking structure 550 comprises a deformable overhang button L21 and a latch member L23 and utilizes the cooperation of these two members to lock and release the device. The locking structure 550 includes a deformable overhang button L21 located at an edge of the front frame 520, and the rear frame 530 includes a complementary groove so that the housing 500 has a smooth and complete appearance when it is closed. The deformable overhang button L21 is attached to a latch member L23 on the inner side of the button L21, and the latch member L23 comprises a latch body L233 and a pillar L231 for connecting the button L21 and the latch body L233. Two semi-open notches L232 (between the button L21 and the latch body L233) are formed by the button L21, the pillar L231 and latch body L233. Two protrusions L24 extend from the inner surface of the rear frame 530, at positions corresponding to the notches L232. When the housing 500 is closed, the protrusions L24 enter the notches L232, and at the same time, the pillar L231 is stuck in the gap between the protrusions L24, providing a stable lock between the front frame 520 and the rear frame 530. In order to open the housing 500 again, the consumer may press the deformable overhang button L21, to make deformation of the button, so that the pillar L231 breaks away from the gap and the protrusions L24 detach from the notches, thereby unlocking the latch structure. Also shown in FIG. 5C are two rib elements 570 that are configured to impact the membrane 340 of the cartridge when the housing 500 is closed. Thus, the rib elements will apply a compressive force to the cartridge when the housing 500 is closed, which provides advantages discussed herein. As will be appreciated by a person skilled in the art, the cartridge is of an appropriate size for the closing of the housing to generate a required compressive force for the advantages discussed herein to be obtained.

The front frame 520 is shown in more detail in FIG. 5D. The front frame 520 comprises a window 580 that is configured to receive a part of the cartridge. A part of the perimeter of the window 580 is bordered by a wall portion 581 protruding substantially perpendicular from the window 580 towards an interior of the housing. The wall portion 581 is configured to support the cartridge in the housing without adhesion. The deformable overhang button L21 discussed above is also visible.

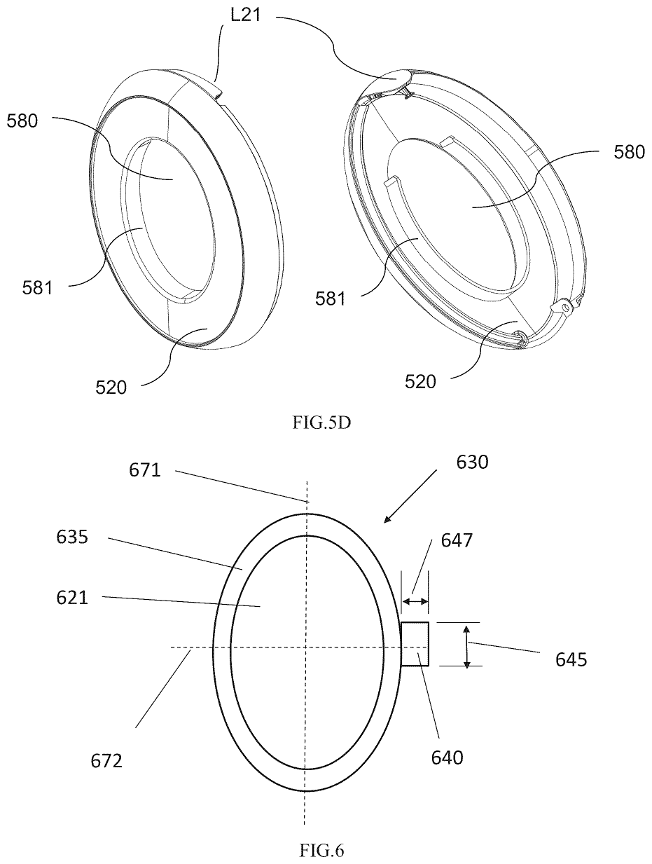

FIG. 6 depicts a cartridge 630 in accordance with the present disclosure. The cartridge 630 comprises a seal area 635 which, as noted herein may comprise a portion of the sealing substrate and a portion of the membrane. In order to facilitate removal of the sealing substrate a pull tab 640 may be provided. The pull tab 640 may be integrally formed with the sealing substrate or may be discrete and joined to the sealing substrate. The cartridge may comprise a major axis 671 and a minor axis 672. The pull tab 640, in order to facilitate packaging may have a length 645 generally parallel to the long axis 671 and a width 647 generally parallel to the minor axis 672. The length 645 and width 647 of the pull tab 640 may be any suitable value as described herein.

FIG. 7 shows an illustrated side cross-section of a cartridge 701, having a reservoir 704 containing volatile composition. The reservoir 704 is enclosed by a membrane 703a. The cartridge 701 is not subjected to any compressive force, so the membrane 703a is not deformed, and the reservoir has a fill level of volatile composition 704a. In contrast, the cartridge 702 is subjected to a compressive force F acting in a horizontal direction. The compressive force F deforms the membrane 703b of the cartridge, thereby reducing a horizontal thickness of the reservoir 704, causing an increase in the fill level of volatile composition 704b.

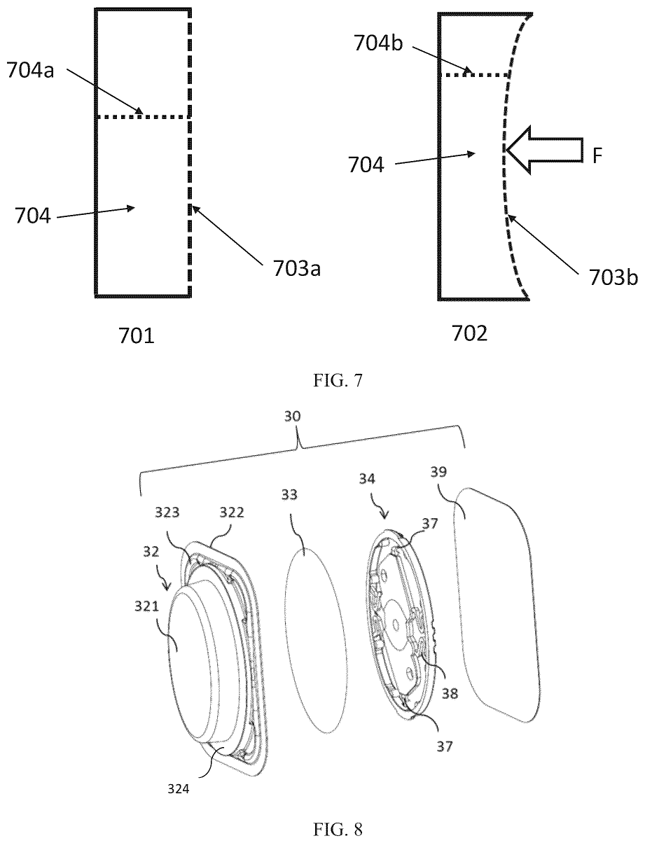

FIG. 8 shows an expanded view of a cartridge 30 according to a configuration. The cartridge 30 comprises a reservoir 32 having a main reservoir body 321, within which the volatile composition is stored. A sealing substrate 33 is sealably attached to and encloses the reservoir 32 at an inner peripheral seal area 324 to prevent the volatile composition from being released until the cartridge 30 is activated. The sealing substrate 33 may be ruptured to release the volatile composition by actuating a rupture mechanism 34 positioned adjacent to the sealing substrate 33. One or more rupture elements 37 are arranged within the rupture mechanism 34 to puncture holes in the sealing substrate 33. The rupture element 37 may be a pin. The cartridge 30 may comprise a membrane 39 located on the exterior of the cartridge 30. The membrane 39 may be scalably attached to an outer peripheral seal area 322 located at a peripheral seal area 323 of the reservoir 32. The peripheral seal area 323 also comprises the inner peripheral seal area 324 in which the sealing substrate 33 is sealably attached to the reservoir 32. The membrane 39 may enclose the reservoir 32, the volatile composition, the sealing substrate 33, and the rupture mechanism 34. The membrane 39 may be configured to flex when a pressure or an actuation force is applied on the membrane 39. And the membrane 39 may be configured to porous so as to sustainably absorb and volatilize the volatile composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any examples disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular configurations of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A consumer product comprising:
(i) an outer packaging;
(ii) one or more cartridges containing a volatile composition; and
(iii) instructions for using the one or more cartridges with a reusable housing;
wherein the one or more cartridges are packaged within the outer packaging, and each comprise:
a reservoir for interfacing with the reusable housing, the reservoir containing a volatile composition in liquid form and having an opening;
a membrane enclosing the opening of the reservoir, the membrane configured to allow evaporation of the volatile composition; and
a sealing substrate enclosing the opening of the reservoir,
wherein:
the one or more cartridges are single-use cartridges for placing into the reusable housing; and
the one or more cartridges are configured to be placed into the reusable housing without adhesion or attachment to the reusable housing; and
either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

2. A consumer product comprising:
(i) an outer packaging;
(ii) one or more cartridges containing a volatile composition; and
(iii) instructions for using the one or more cartridges with a reusable housing;
wherein the one or more cartridges are packaged within the outer packaging, and each comprise:
a reservoir for interfacing with the reusable housing, the reservoir containing a volatile composition in liquid form and having an opening;
a membrane enclosing the opening of the reservoir, the membrane configured to allow evaporation of the volatile composition; and
a sealing substrate enclosing the opening of the reservoir,
wherein:
the one or more cartridges are single-use cartridges for placing into the reusable housing; and
the one or more cartridges are configured to be removed from the reusable housing without being touched by a user; and
either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

3. The consumer product according to claim 1, wherein the sealing substrate encloses the membrane, and the sealing substrate is configured to be removed before use.

4. The consumer product according to claim 1, wherein the one or more cartridges comprise an outer peripheral seal area, and wherein a peripheral portion of the sealing substrate and membrane are sealed together at the outer peripheral seal area,
wherein the membrane is sealed to a peripheral portion of the reservoir at the outer peripheral seal area.

5. The consumer product according to claim 1, wherein the sealing substrate comprises a pull tab to facilitate removal by a user.

6. The consumer product according to claim 1, wherein the volatile composition comprises a perfume.

7. The consumer product according to claim 1, wherein the volatile composition comprises a volatile carbonyl containing compound having a vapor pressure of at least 0.025 torr at 25 degrees Celsius, wherein the volatile carbonyl containing compound is selected from the group consisting of: volatile aldehydes, ketones, and mixtures thereof.

8. The consumer product according to claim 1, wherein the one or more cartridges comprise at least one secondary reservoir portion,
wherein when the cartridge is oriented for use:
at least one secondary reservoir portion is gravitationally below the reservoir; and
a total volume of the at least one secondary reservoir portion that is gravitationally below the primary reservoir portion is at least 4% of the initial volume of volatile composition.

9. The consumer product according to claim 1, wherein the reservoir is an outermost layer of the one or more cartridges.

10. The consumer product according to claim 1, wherein the one or more cartridges are compressible or deformable.

11. The consumer product according to claim 10, wherein the membrane and reservoir are each compressible or deformable.

12. The consumer product according to claim 1, wherein the membrane has an evaporative surface area of from about 20 cm² to about 40 cm², and the one or more cartridges have a maximum dimension of less than 10 cm.

13. The consumer product according to claim 1, wherein the membrane forms substantially all of the surface area of a face of the cartridge,
wherein the membrane forms at least 90% of the surface area of a face of the cartridge,
wherein the membrane forms at least 95% of the surface area of a face of the cartridge.

14. The consumer product according to claim 1, wherein the reservoir is formed from a transparent material to enable viewing of a fill level of volatile composition.

15. The consumer product according to claim 1, wherein the reusable housing comprises one or more rib elements that are configured to apply a compressive force to a cartridge placed within the reusable housing, where the compressive force is configured to be applied to the cartridge for substantially as long as the cartridge is present within the reusable housing.

16. The consumer product according to claim 1, wherein the reusable housing comprises a front frame and a rear frame that are capable of moving away from or toward each other for opening and closing the reusable housing,
wherein:
the front frame comprises a window configured to receive a part of the cartridge (e.g. the reservoir);
at least a part of a perimeter of the window is bordered by a wall portion protruding substantially perpendicular from the window towards an interior of the reusable housing, the wall portion configured to support the cartridge (e.g. the reservoir) within the reusable housing without adhesion;
the rear frame comprises one or more apertures configured to allow an airflow into the reusable housing; and
the reusable housing is configured to hold the cartridge between the front and rear frames so as to allow the volatile composition to evaporate from the cartridge.

17. The consumer product according to claim 1, wherein the reusable housing comprises a front frame and a rear frame that are capable of moving away from or toward each other for opening and closing the reusable housing, wherein the rear frame comprises one or more rib elements that are configured to apply a compressive force on the cartridge when the reusable housing is in a closed state and the cartridge is positioned within the reusable housing, where the compressive force is configured to be applied to the cartridge for substantially as long as the cartridge is present within the reusable housing.

18. The consumer product according to claim 17, wherein the compressive force is configured to cause a reduction in a thickness of the reservoir of the cartridge when held within the reusable housing, as compared to a thickness of the reservoir before being subjected to the compressive force.

19. A consumer product comprising:
(i) an outer packaging; and
(ii) one or more cartridges containing a volatile composition, the one or more cartridges being single-use cartridges for placing into a reusable housing and packaged within the outer packaging, and each comprising:
a reservoir for interfacing with the reusable housing, the reservoir containing a volatile composition in liquid form and having an opening;
a membrane enclosing the opening of the reservoir, the membrane configured to allow evaporation of the volatile composition; and
a sealing substrate enclosing the opening of the reservoir,
wherein:
the one or more cartridges are configured such that, when placed into a housing comprising a front frame and a rear frame that are capable of moving away from or toward each other for opening and closing the housing, wherein: the front frame comprises a window configured to receive a part of the cartridge (e.g. the reservoir); at least a part of a perimeter of the window is bordered by a wall portion protruding substantially perpendicular from the window towards an interior of the housing, the wall portion configured to support the cartridge (e.g. the reservoir) within the housing without adhesion; the rear frame comprises one or more apertures configured to allow an airflow into the housing; and the housing is configured to hold the cartridge between the front and rear frames so as to allow the volatile composition to evaporate from the cartridge, said cartridges are held within the reusable housing without adhesion or attachment to the reusable housing and may be removed from the reusable housing without being touched by a user; and
either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.
